(19) United States
(12) Patent Application Publication
(10) Pub. No.: US 2022/0184609 A1
(43) Pub. Date: Jun. 16, 2022

(54) MICROPLATE FOR MICROSCOPY

(71) Applicant: Phase Holographic Imaging PHI AB, LUND (SE)

(72) Inventors: Peter EGELBERG, LUND (SE); Jonas BERGKVIST, Bjärred (SE)

(21) Appl. No.: 17/598,481

(22) PCT Filed: Mar. 27, 2020
(86) PCT No.: PCT/EP2020/058836

§ 371 Date: Sep. 27, 2021

(30) Foreign Application Priority Data
Mar. 29, 2019 (SE) 1950396-0

(51) Int. Cl. B01L 3/00; G01N 21/64; G01N 21/03; G06T 7/00

(52) U.S. Cl. CPC B01L 3/5085; G01N 21/6452; G01N 21/6458; G01N 21/03; G06T 7/0012; G06T 2207/30024; B01L 2300/0829; G01N 2021/0367; G06T 2207/10056; G06T 2207/10064; B01L 2300/168

(57) ABSTRACT

The invention relates to a microplate (10) for microscopy of an organoid (30), comprising: a body (11) having at least one recess (20), the recess (20) being adapted to contain and restrict movement of the organoid (30), and a reflective surface (40), the reflective surface (40) being inclined in relation to the recess (20), such that an incoming beam of light towards the microplate (10) is directed onto the organoid (30) in a substantially horizontal direction.

### FIELD OF THE INVENTION

The invention relates to a microplate for microscopy of an organoid and to a method for microscopy of an organoid.

### BACKGROUND OF THE INVENTION

A common container for growing cells, such as in an organoid, is called a microplate. Microplates are flat plates, most often made of plastic, which have a plurality of wells arranged in a matrix on them. Each well may typically hold between several nanoliters and several milliliters of liquid and are used as small test tubes in which cells may be cultured. Typical microplates may have anywhere between 6 and 1536 wells arranged in a rectangular matrix. The wells are typically formed as cylinders in the microplate having an open top and a closed bottom such that they may hold a small amount of liquid.

When growing cells in a well of a microplate it is desirable to be able to monitor the cells, for instance to monitor the cell growth or the current number of cells. The microplate may thus be placed in a microscope such that the cells in the well are observed either from above or from below the well, most often in a direction along the depth extension of the well. For this reason, the microplates, or at least the portion of the microplate which is situated under a well, are made transparent such that the cells in a well can be monitored by a microscope.

Since most cells are transparent it may be difficult to monitor them using a regular optical microscope. In order to better observe the cells and cellular structures, the cells and cellular structures may be colored prior to being observed. However, such colorings often result in the death of the cells which prevents phenomena of living cells from being observed.

An alternative technique is to attach fluorescent molecules to living cells. Then by directing an excitation beam of light with a suitable wavelength towards the cells the fluorescent molecules will be excited and send out colored light of their own which light may be observed in a microscope. Typically, the excitation beam of light is a laser beam. An advantage of attaching fluorescent molecules is that it allows for the monitoring of just a portion of the cells by only illuminating that portion with the excitation beam of light.

A problem with conventional microplates is that they only allow vertical illumination, and thus excitation, of the fluorescent molecules. Thus it will only be possible to observe portions of the cells which extend in a vertical direction.

It is not possible to illuminate, and thus excite the fluorescent molecules of, portions of the cells which extend in a horizontal direction, that is a direction which is perpendicular to the imaging axis. This as the microplate itself is in the way of the particular well of interest.

Thus, improvements to microplates which allows for horizontal illumination of cells in a well is desired.

### SUMMARY OF THE INVENTION

It is an object of the invention to provide a microplate for microscopy of an organoid which allows for horizontal illumination of the organoid.

According to a first aspect, these and other objects are achieved in full, or at least in part, by a microplate for microscopy of an organoid, comprising

a body having at least one recess, the recess being adapted to contain and restrict movement of the organoid, and

a reflective surface,

wherein the reflective surface is inclined in relation to the recess, such that an incoming beam of light towards the microplate is directed onto the organoid in a substantially horizontal direction.

By such a microplate, it is possible to illuminate an organoid situated in the microplate from a horizontal direction. Thus, it is possible to selectively excite fluorescent molecules of a portion of the organoid which extends in a horizontal direction. If the excitation beam of light is in the form of a light-sheet it is possible to selectively excite a sheet of the organoid extending in horizontal direction. This allows for an image of a sheet of the organoid extending in the same plane as an image acquired by regular vertical illumination microscopy of the organoid. Thus, the microplate allows for the cultivation of cells, such as an organoid, and for both fluorescent microscopy and regular vertical illumination microscopy. There is no need to move the organoid out of the microplate in order to be able to monitor it by microscopy, especially for fluorescent microscopy.

The beam of light may be a laser beam. A laser beam is a suitable beam of light for exciting fluorescent molecules.

The incoming beam of light may be incoming in a vertical direction.

The incoming beam of light may be incoming at the top of the microplate. The incoming beam of light may be incoming at the bottom of the microplate.

The reflective surface may be formed between a first volume having a first refractive index and a second volume having a second refractive index.

By this, the reflective surface may direct an incoming beam of light towards the organoid by reflection. Preferably, the conditions are met for the beam of light to be directed towards the organoid by total internal reflection. To meet the conditions for total internal reflection the quota of the refractive indexes have to be large enough and the incoming angle of the incoming beam of light has to be large enough.

The first refractive index may be larger than the second refractive index.

By this, the reflection of a beam of light traveling in the first volume incoming towards the second volume may be facilitated.

The first volume may be a portion of the body having a first refractive index and the second body may be a portion of the body having a second refractive index.

By this, the reflective surface may be made from the body itself. For instance, by forming a structure in the body with a different refractive index than the main part of the body at which structure a beam of light may be reflected.

The first volume may be made of a first material and the second volume may be made of a second material.

By this, the reflective surface may be made by forming a structure in the body of a different material from the main part of the body, having a different refractive index than the main part of the body, at which structure a beam of light may be reflected.

The first volume may be a portion of the body and the second volume may be constituted by surrounding medium present in a groove in the bottom of the body.

By this, the reflective surface may be achieved in a simple and way, without any complicated formations of structures in the body itself.

The groove may be formed in a triangular shape.

This allows for a groove having a shape which is particularly suitable for directing an incoming beam of light towards an organoid regardless of where the beam of light hits the groove. Further, this shape may allow for the reflection of a beam of light on at least two sides of the groove, thus allowing the groove to be used for more than one well of a microplate.

The recess may be formed as cylinder or as a prism.

This is a shape which is suitable both for culturing cells in and for microscopy of the cells.

Another shape which is possible is the shape of a cone or a pyramid where the apex of the cone or pyramid is positioned towards the bottom side of the microplate. The cone or pyramid may or may not be truncated.

The reflective surface may be arranged such that each point of the organoid may be illuminated by a beam of light incoming towards a corresponding point of the reflective surface.

By this, the microplate allows for the selective horizontal illumination of every part of the organoid. The organoid may be horizontally illuminated all at once or selected portions of the organoid may be horizontally illuminated. The organoid may be horizontally illuminated in its entirety portion by portion by directing a beam of light towards corresponding portions of the reflective surface, portion by portion.

By “corresponding point” of the reflective surface is here meant a point on the reflective surface which directs an incoming beam of light towards the point on the organoid in a horizontal direction.

The reflective surface may be inclined relative the recess by 45 degrees.

By this, the reflective surface may direct an incoming beam of light, which is incoming in a vertical direction, towards the organoid in a horizontal direction.

The reflective surface may be inclined relative the recess by between 40 and 50 degrees. This will allow the reflective surface to direct an incoming beam of light, which is incoming in a direction close to a vertical direction, towards the organoid in a horizontal direction.

The recess may comprise a first portion being adapted to contain and restrict movement of the organoid by having a horizontal cross sectional area such that the movement of the organoid is restricted.

By this, the organoid may be kept in place such that images of it can be acquired.

The recess may comprise a second portion being adapted to receive a pipette,

wherein the second portion has a horizontal cross sectional area such that it may receive a pipette.

By this, the environment of the cell culture may be easily accessed and for instance, nutrition can be provided to the cells.

The second portion may have a larger horizontal cross sectional area than the first portion. Each of the first portion or second portion or both may have the shape of a prism or cylinder.

The recess may comprise a third portion connecting the first portion and the second portion, wherein the third portion may be formed as a truncated cone or truncated pyramid.

The body may be made of a transparent material.

This will allow the cells to be monitored by microscopy as light may pass through the body.

The body may be made of polystyrene. Polystyrene is a suitable material which can be made transparent.

According to a second aspect there is provided a method for microscopy of an organoid in a microplate, the microplate comprising:

a body having at least one recess, the recess being adapted to contain and restrict movement of the organoid, and

a reflective surface, the reflective surface being inclined in relation to the recess, such that an incoming beam of light towards the microplate is directed onto the organoid in a substantially horizontal direction

the method comprising the steps of:

placing the organoid in the recess,

illuminating a portion of the organoid from a substantially horizontal direction by sending a beam of light onto the reflective surface of the microplate,

capturing the light signal from the illuminated portion of the organoid to form an image of the illuminated portion of the organoid.

By such a method it is possible to horizontally illuminate an organoid in a microplate. Thus, it is possible to horizontally excite fluorescent molecules attached to an organoid and acquire fluorescent images of portions of the organoid extending in a horizontal direction.

The method may further comprise the steps of:

stepwise illuminating a plurality of portions of the organoid from a substantially horizontal direction by stepwise sending a beam of light onto a corresponding portion of the reflective surface of the microplate, and

stepwise capturing the signal from the plurality of illuminated portions of the organoid to form an image of the plurality of illuminated portions of the organoid.

By this, a fluorescent image of several distinct portions of the organoid extending in a horizontal direction can be acquired. Thus a three-dimensional image of the organoid can be constructed by adding the images together.

The microplate has a bottom side and a top side being positioned at opposite sides of the microplate from each other. When placing the microplate at ground the bottom side of the microplate is the side which is to be facing ground. The top side are the side at which the recesses have their opening. Each recess at least partly extends from the top side towards the bottom side. The bottom side is the side which is to be placed such that anything which is placed in the recess is kept there by gravity.

The microplate has a height defined as a direction extending between the top side and the bottom side.

A recess has a depth defined as the direction along the recess from the top side of the microplate, that is the opening of the recess, and the bottom of the recess.

By a horizontal direction is here meant a direction which is perpendicular to an observation direction at which an organoid sample in the microplate is observed by microscopy.

In other words, a horizontal direction is a direction which is perpendicular to the imaging axis.

For microscopy the observation direction is the direction at which the signal of a sample is captured by an objective.

For fluorescence microscopy observation direction is the direction at which the fluorescent signal of a sample is captured by an objective.

The observation direction is parallel to the extension of the recess along the depth of the recess. The observation direction may also be called a vertical direction.

By cylinder and prism is here meant a shape which has an equal base and top area and straight parallel sides connecting the base and top area. Other shapes, having other types of base and top areas, like this are imaginable which we here include in the terms cylinder or prism.

### BRIEF DESCRIPTION OF THE DRAWINGS

The above objects, as well as additional objects, features and advantages of the present invention, will be more fully appreciated by reference to the following illustrative and non-limiting detailed description, when taken in conjunction with the accompanying drawings.

FIG. 1 shows a perspective view of a microplate.

FIG. 2 shows a vertical cross sectional view of a portion of a microplate.

FIG. 3 shows a vertical cross sectional view of a portion of a microplate.

FIG. 4 shows a vertical cross sectional view of a portion of a microplate.

### DETAILED DESCRIPTION OF THE INVENTION

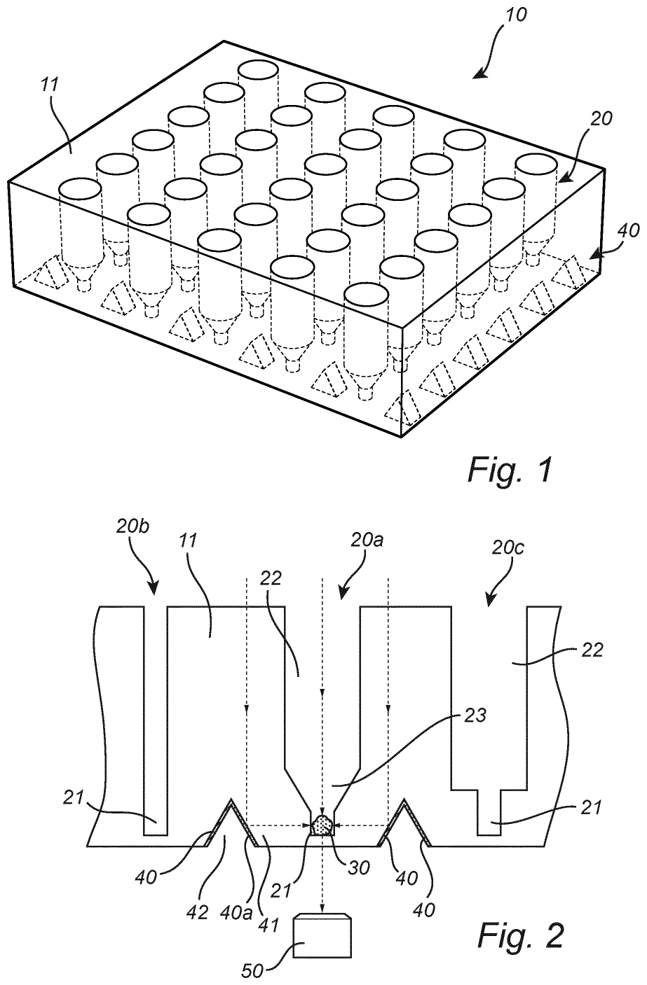

In FIG. 1 a microplate 10 can be seen. The microplate 10 is for microscopy of an organoid 30.

The microplate 10 has the shape of a cuboid. Other shapes are also possible, for example a cylindrical shape.

The microplate comprises a body 11. The body 11 has a plurality of recesses 20 in the form of wells 20. The body 11 may have just one recess 20 or a plurality of recesses 20.

The body 11 is made of a transparent material. The material at least being transparent to the light being used for microscopy. The body 11 may be made of transparent polystyrene.

The recesses 11 extend from a top side of the microplate 10 towards a bottom side of the microplate 10. The recesses 11 are open on the top side of the microplate 10, but do not extend through the entire height of the microplate 10.

In FIG. 1 the recesses 20 are arranged in a matrix on the microplate 10. Other arrangements are also possible, ordered or non-ordered.

The recesses 11 are each adapted to contain and restrict movement of an organoid 30.

The microplate 10 comprises a reflective surface 40. As can be seen in FIG. 1 the microplate 10 may comprise a plurality of reflective surfaces 40. As can be seen in FIG. 1 there may be a reflective surface 40 next to each recess 20. There may be more than one reflective surface 40 next to each recess 20.

The term “next to” here means that there is no structure between the reflective surface 40 and the recess 20 which hinders light passing between the reflective surface 40 and the recess 20. By hinder is here meant that the light is blocked, absorbed or directed away from the recess 20.

A reflective surface 40 may extend for longer than what is seen in FIG. 1. A reflective surface 42 may extend such that it forms a reflective surface 40 for a number of recesses 20. Several reflective surfaces 40 in FIG. 1 may be connected into one longer reflective surface 40. A reflective surface 40 may extend alongside the entire length or width of the microplate 10. A reflective surface 40 may extend for up to 90% of the length or width of the microplate 10.

In FIGS. 2 and 4 dashed lines represent beams of light and how beams of light travel with respect to the microplate 10.

In FIG. 2 a vertical cross sectional view of a portion of a microplate 10 is seen. A plurality of recesses 20 can be seen. An organoid 30 is contained by a recess 20a. It is possible to have more than one organoid 30 contained in the same recess 20. The recess 20a restricts the movement of the organoid 30. Preferably a horizontal width of the recess 20 is larger than the width of the organoid 30 while still being small enough that the movement of the organoid 30 is restricted. Preferably this means that a horizontal width of the recess 20 is at maximum 10 times larger than the width of the organoid 30.

A horizontal width of the recess 20 may be large enough that the organoid 30 isn't squeezed in place by the sidewalls of the recess 20. In other words, a horizontal width of the recess 20 may be larger than the horizontal width of the organoid 30.

A horizontal width of the recess 20 may be between 2 and 3 times larger than the horizontal width of the organoid 30.

A horizontal width of the recess 20 may be between 1 and 10 times larger than the horizontal width of the organoid 30.

A horizontal width of the recess 20 may be between 1.5 and 10 times larger than the horizontal width of the organoid 30.

A horizontal width of the recess 20 may be between 3 and 8 times larger than the horizontal width of the organoid 30.

A horizontal width of the recess 20 may be between 1.1 and 2 times larger than the horizontal width of the organoid 30.

A contained organoid 30 may have a width of 10-10 000 micrometers. A horizontal cross sectional width of the recess 20 may thus be between 10 and 100 000 micrometers.

In FIG. 2 an objective 50 is seen by which the organoid 30 is observed. The objective 50 is adapted to capture light from the organoid 30 to form an image of the organoid 30. The objective 50 observes and captures light from the organoid 30 in a vertical direction, in other words along an imaging axis.

In FIG. 2 a reflective surface 40a can be seen. The reflective surface 40a is inclined in relation to the recess such that an incoming beam of light towards the microplate is directed onto the organoid in a substantially horizontal direction.

The reflective surface 40 is arranged such that each point of the organoid may be illuminated by a beam of light incoming towards a corresponding point of the reflective surface.

The quota between the first refractive index and the second refractive index may be such that an incoming beam of light towards the microplate (10) is directed onto the organoid (30) in a substantially horizontal direction by total internal reflection.

In FIG. 2 the reflective surface 40 is formed between a first volume 41 having a first refractive index and a second 42 volume having a second refractive index. The first refractive index being larger than the second refractive index.

In FIG. 2 the first volume 41 is a portion of the body itself 11. The second volume 42 is constituted by surrounding medium present in a groove 42 in the bottom of the body 11.

A single groove 42 may extend such that it forms a reflective surface 40 for a number of recesses 20. A groove 42 may extend for the entire length or width of the microplate 10. A groove 42 may extend for up to 90% of the length or width of the microplate 10.

The groove 42 is formed in a triangular shape. The groove 42 may be formed in a different shape such as a curved shape or another polygonal shape. The groove 42 may be formed such that the reflective surface 40 is a flat surface as this simplifies the direction of incoming light onto the organoid 30. A polygonal shape is a shape which is suitable for producing a flat reflective surface 40.

If the incoming beam of light is incoming at the bottom of the microplate 10 the groove 42 may have a first side for reflecting the beam of light and another side inclined such that the beam of light enters the body 11 such that the beam of light is directed onto the organoid 30 in a substantially horizontal direction.

As an alternative, the first volume 41 may be a portion of the body 11 itself having the first refractive index and the second volume 42 may be a portion of the body 11 itself having the second refractive index. The first volume 41 may then be made of a first material and the second volume 42 may be made of a second material.

As can be seen in FIG. 2, a recess 20, 20a, 20b, 20c may have different shapes.

A recess 20 may comprise a first portion 21 being adapted to contain and restrict movement of the organoid 30 by having a horizontal cross sectional area such that the movement of the organoid 30 is restricted. Preferably the horizontal width of the first portion 21 is larger than the width of the organoid 30 while still being small enough that the movement of the organoid 30 is restricted. Preferably this means that the horizontal width of the first portion 21 is at maximum 10 times larger than the width of the organoid 30.

The horizontal width of the first portion 21 may be between 2 and 3 times larger than the horizontal width of the organoid 30.

The horizontal width of the first portion 21 may be between 1 and 10 times larger than the horizontal width of the organoid 30.

The horizontal width of the first portion 21 may be between 1.5 and 10 times larger than the horizontal width of the organoid 30.

The horizontal width of the first portion 21 may be between 3 and 8 times larger than the horizontal width of the organoid 30.

The horizontal width of the first portion 21 may be between 1.1 and 2 times larger than the horizontal width of the organoid 30.

A contained organoid 30 may have a width of 10-1000 micrometers. The horizontal cross sectional width of the first portion 21 may thus be between 10 and 10 000 micrometers.

As can be seen in FIG. 2 a recess 20b may formed in a way corresponding to only comprising a first portion 21, that is the horizontal cross section of the entire recess 20b may be such that it is adapted to contain and restrict movement of the organoid 30 by having a horizontal cross sectional area such that the movement of the organoid 30 is restricted.

As can be seen in FIG. 2, a recess 20a, 20c may comprise a second portion 22. The second portion 22 being adapted to receive a pipette. The second portion 22 has a horizontal cross sectional area such that it may receive a pipette.

As can be seen in FIG. 2, a recess 20a may comprise a third portion 23. The third portion connecting the first portion 21 and the second portion 22. The third portion 23 may connect the sidewalls of the first portion 21 with the sidewalls of the second portion 22. The third portion 23 may have slanting sidewalls such that the sidewalls of the first portion 21 and the sidewalls of the second portion 22 may be connected through the third portion 23.

The horizontal cross section of a recess 20 may take the shape of a polygon or a curved shape, such as a circle. Thus a recess 20 may have a shape of a cylinder or of a prism.

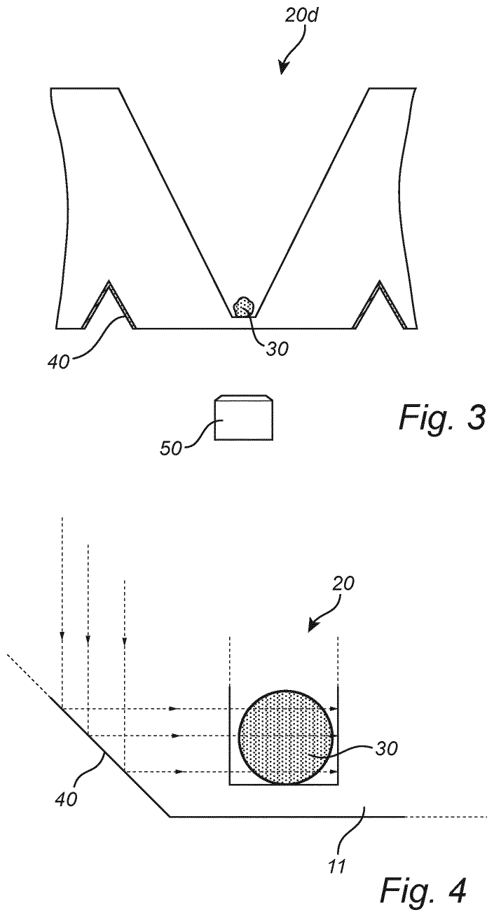

As can be seen in FIG. 3, a recess 20 may have the shape of a cone or a pyramid. In FIG. 3 the apex of the cone or pyramid is positioned towards the bottom of the microplate 10. The cone or pyramid shape may or may not be truncated.

In FIGS. 2 and 3, it can be seen that the recess 20 doesn't extend all the way through the body 11. Beneath the recess 20, that is towards the bottom side of the microplate 10, the microplate 10 is left intact by a thin thickness. This thickness may be between 10 and 100 micrometers. This is a thickness which is suitable for allowing light from the organoid 30 to be captured by an objective 50.

In FIG. 4, it is illustrated how different portions of the organoid 30 can be illuminated by an incoming light beam. The incoming light beam will be directed by the reflective surface 40 onto a portion of the organoid 30 in a substantially horizontal direction. It is thus possible to illuminate the entire organoid 30 from a horizontal direction. It is also possible to illuminate various specific portions of the organoid 30 in this way. Several portions of the organoid 30 may be stepwise illuminated in this way. Allowing for a composite three-dimensional image of the organoid 30 to be constructed from the stepwise captured images.

A method for microscopy of an organoid 30 in a microplate 10 such as one described above will now be described.

The method comprises placing the organoid 30 in the recess 20. The method comprises illuminating a portion of the organoid 30 from a substantially horizontal direction by sending a beam of light onto the reflective surface 40 of the microplate 10.

The beam of light may be in the form of a light sheet. The illuminated portion of the organoid 30 will then be in the form of a sheet of the organoid 30. The sheet extending in a horizontal direction.

The method comprises capturing the light signal from the illuminated portion of the organoid 30 to form an image of the illuminated portion of the organoid.

These steps may be repeated such that images of a plurality of portions of the organoid 30 can be captured. This by redirecting the beam of light onto a different portion of the reflective surface 40 which will direct the beam of light onto a different portion of the organoid 30.

By capturing images of a plurality of portions of the organoid 30 a three-dimensional composite image of the organoid can be constructed.

If the beam of light is in the form of a light sheet every step will capture illuminate a sheet of the organoid 30, and thus produce an image of that sheet of the organoid 30. By stepwise directing the beam of light to a portion of the reflective surface 40 next to or even overlapping the previous portion of the reflective surface to which the beam of light was directed it is possible to illuminate, and thus produce a three-dimensional image of, continuous portions of the organoid 30, and even the entire organoid 30.

The method may thus comprise stepwise illuminating a plurality of portions of the organoid 30 from a substantially horizontal direction by stepwise sending a beam of light onto a corresponding portion of the reflective surface 40 of the microplate 10, and

stepwise capturing the signal from the plurality of illuminated portions of the organoid 30 to form an image of the plurality of illuminated portions of the organoid 30.

1. A microplate for microscopy of an organoid, comprising:
a body having at least one recess, the recess being adapted to contain and restrict movement of the organoid, and
a reflective surface,
characterized in that the reflective surface is inclined in relation to the recess, such that an incoming beam of light towards the microplate is directed onto the organoid in a substantially horizontal direction.

2. A microplate according to claim 1, wherein the reflective surface is formed between a first volume having a first refractive index and a second volume having a second refractive index.

3. A microplate according to claim 2, wherein the first refractive index is larger than the second refractive index.

4. A microplate according to claim 2, wherein the first volume is a portion of the body having a first refractive index and the second volume is a portion of the body having a second refractive index.

5. A microplate according to claim 2, wherein the first volume is made of a first material and the second volume is made of a second material.

6. A microplate according to claim 2, wherein the first volume is a portion of the body and the second volume is constituted by surrounding medium present in a groove in the bottom of the body.

7. A microplate according to claim 6, wherein the groove is formed in a triangular shape.

8. A microplate according to claim 1, wherein the recess is formed as cylinder or as a prism.

9. A microplate according to claim 1, wherein
the reflective surface is arranged such that each point of the organoid may be illuminated by a beam of light incoming towards a corresponding point of the reflective surface.

10. A microplate according to claim 1, wherein
the reflective surface is inclined relative the recess by 45 degrees.

11. A microplate according to claim 1, wherein
the recess comprises a first portion being adapted to contain and restrict movement of the organoid by having a horizontal cross sectional area such that the movement of the organoid is restricted.

12. A microplate according to claim 11, wherein the recess comprises:
a second portion being adapted to receive a pipette,
wherein the second portion has a horizontal cross sectional area such that it may receive a pipette.

13. A microplate according to claim 1 wherein
the body is made of a transparent material.

14. Method for microscopy of an organoid in a microplate, the microplate comprising:
a body having at least one recess, the recess being adapted to contain and restrict movement of the organoid, and
a reflective surface, the reflective surface being inclined in relation to the recess, such that an incoming beam of light towards the microplate is directed onto the organoid in a substantially horizontal direction
characterized by the steps of:
placing the organoid in the recess,
illuminating a portion of the organoid from a substantially horizontal direction by sending a beam of light onto the reflective surface of the microplate,
capturing the light signal from the illuminated portion of the organoid to form an image of the illuminated portion of the organoid.

15. Method according to claim 14, further comprising the steps of:
stepwise illuminating a plurality of portions of the organoid from a substantially horizontal direction by stepwise sending a beam of light onto a corresponding portion of the reflective surface of the microplate, and
stepwise capturing the signal from the plurality of illuminated portions of the organoid to form an image of the plurality of illuminated portions of the organoid.
